SEQUENCE LISTING (11 sequences)

SEQ ID NO: 1 - DNA, 67 bp, Artificial
  Primer
tctagtacctgcttcactgcatctaatacgactcactatagggagattttttttttttttttttttt

SEQ ID NO: 2 - DNA, 12 bp, Artificial
  Transcription Reagent
tttttttttttt

SEQ ID NO: 3 - DNA, 13 bp, Artificial
  Transcription Reagent
ttttttttttttt

SEQ ID NO: 4 - DNA, 14 bp, Artificial Sequence
  Transcription reagent
tttttttttttttt

SEQ ID NO: 5 - DNA, 15 bp, Artificial Sequence
  Transcription reagent
ttttttttttttttt

SEQ ID NO: 6 - DNA, 16 bp, Artificial Sequence
  Transcription reagent
tttttttttttttttt

SEQ ID NO: 7 - DNA, 17 bp, Artificial Sequence
  Transcription reagent
ttttttttttttttttt

SEQ ID NO: 8 - DNA, 18 bp, Artificial Sequence
  Transcription reagent
tttttttttttttttttt

SEQ ID NO: 9 - DNA, 67 bp, Artificial Sequence
  Transcription reagent
ttccagtgagtcgtatctaaaactaatacgactcactatagggagattttttttttttttttttttt

SEQ ID NO: 10 - DNA, 28 bp, Artificial Sequence
  Transcription reagent
gctgactcgtactcgagttagtggtagt

SEQ ID NO: 11 - DNA, 20 bp, Artificial Sequence
  Transcription reagent
aaaaaaaaaaaaaaaaaaaa

(19) United States
(12) Patent Application Publication
(10) Pub. No.: US 2006/0105331 A1
(43) Pub. Date: May 18, 2006

(54) Methods for improving rna transcription reactions

(76) Inventors: Fredrik Carl Kamme, San Diego, CA (US); Jessica Y. Zhu, San Diego, CA (US)

Correspondence Address: Philip S Johnson;Chief Patent Counsel, Johnson & Johnson, One Johnson & Johnson Plaza, New Brunswick, NJ, 08933-7003, US

(21) Appl. No.: 10/514,349

(22) PCT Filed: May 30, 2003
(86) PCT No.: PCT/US03/17103

§ 371 Date: Nov. 12, 2004

Related U.S. Application Data
(60) Provisional application No. 60/384,454, filed on May 31, 2002.

(51) Int. Cl. C12Q 1/68; C12P 19/34

(52) U.S. Cl. 435/6; 435/91.2

(57) ABSTRACT

Methods are described for eliminating single-stranded oligonucleotides from a sample prior to RNA transcription, thereby reducing non-template derived production of RNA. In one embodiment, a sample containing the template for RNA transcription is treated with one or more exonucleases to remove single-stranded oligonucleotides from the reaction mixture prior to RNA transcription. In another embodiment, the sample containing the template for RNA transcription is contacted with an oligonucleotide complementary to the single-stranded oligonucleotide present in the sample, and allowed to hybridize to form double-stranded oligonucleotides.

### FIELD OF THE INVENTION

The invention relates to methods for improving RNA polymerase based RNA transcription from a polynucleotide template by eliminating single-stranded oligonucleotides from the sample prior to RNA transcription, thereby reducing non-template derived production of RNA.

### BACKGROUND OF THE INVENTION

T7 RNA polymerase based amplification systems amplify nucleic acids by virtue of T7 RNA polymerase transcribing several RNA molecules of a given DNA template carrying the appropriate promoter sequence. Using high yield transcription kits, such as the Ampliscribe kit from Epicentre (Madison, Wis.), more than a thousand RNA copies can be generated from a single template molecule. This high yield is achieved by having an extremely high concentration of T7 RNA polymerase and ribonucleotides, and by incubating the reaction for relatively extended periods of time, i.e., 3-12 hours (h). In such conditions, it has been shown that side reactions can occur (see Amaud-Barbe et al., Nucleic Acids Res (1998) 26:3550; Biebricher and Luce, Embo J (1996) 15:3458; Cazenave and Uhlenbeck, Proc Natl Acad Sci USA (1994) 91:6972; and Triana-Alonso et al., J Biol Chem (1995) 270:6298). These are reactions in which RNA is created, but not by transcription of the double-stranded DNA template. Examples of such reactions are self-priming of RNA molecules, leading to partially double-stranded RNA molecules or chimeric sequences and the creation of RNA molecules capable of self-replication (i.e., RNA molecules that are replicated by T7 RNA polymerase without a DNA intermediate). Such side reactions are undesirable for numerous reasons: a side reaction may compete with the transcription from the intended template, thus reducing amplification efficiency; chimeric sequences may confound expression profiling by methods such as microarray analysis; and side products may make up a significant amount of the final RNA product, thus making measurements of the mass of specific RNA product unreliable.

T7 RNA amplification involves the creation of a double-stranded cDNA template containing a functional T7 RNA polymerase promoter, from RNA, and subsequent transcription. To achieve higher amplification, a second round of T7 RNA amplification can be performed using the RNA produced in the first round as the input RNA. It is known to those experienced in the art that two rounds of T7 RNA amplification suffers from experimental artifacts to a much higher degree than one round of T7 RNA amplification does. This is typically seen as a high molecular weight smear of RNA present in samples, whether or not any RNA was present in the original sample. Depending on the purification methods used in the protocol, a low molecular weight RNA may be seen as well, independent of any starting material.

We have found that some single-stranded oligonucleotides, if present in the T7 RNA transcription mix, will result in the production of RNA by T7 RNA polymerase. This is a template-independent reaction that does not require a T7 promoter sequence or double-stranded DNA. A homopolymeric oligonucleotide consisting of 12-18 deoxythymidine bases present in the transcription mix will result in the production of RNA, whereas an oligonucleotide consisting of 20 deoxyadenosine bases will not. Double-stranded DNA not containing a T7 promoter sequence will not result in the production of RNA. Thus, we have discovered a single-strand-dependent, sequence-dependent, non-template-dependent production of RNA by T7 RNA polymerase. In two rounds of T7 amplification, RNA produced by this mechanism during the transcription reaction in the first round will be amplified in the second round, and may generate large amount of RNA. Therefore it is important to minimize the template-independent production of RNA in the first transcription reaction.

Single-stranded oligonucleotide containing a stretch of deoxythymidine bases may be present in the transcription reaction as a result of carry-over from the initial reverse transcription reaction, which is primed using an oligo-dT primer carrying a T7 promoter sequence in the 5′-end. In the T7 RNA amplification protocol, cDNA is initially synthesized from mRNA. Second-strand cDNA is then synthesized, creating a functional template for T7 RNA transcription. The double-stranded cDNA template is purified and then transcribed in a T7 RNA transcription mix. The oligonucleotide used for priming cDNA synthesis contains a stretch of deoxythymidine bases, usually 21, the T7 core promoter sequence, usually 23 bases, and a stretch of irrelevant ‘buffer’ sequence to protect the 5′ end of the promoter sequence from exonucleolytic digestion, 20-25 bases long. Therefore, the primer (T7dT₂₁) is usually 65 to 70 bases long. Oligonucleotides of this length are inefficiently removed by purification steps such as ethanol precipitation, silica spin columns or Microcon centrifugal purification membranes, such as the YM-100. These are typical purification methods used in T7 RNA amplification. We have developed methods for eliminating single-stranded oligonucleotide from the sample prior to T7 RNA transcription, thus inhibiting the undesired non-template derived production of RNA in the transcription reaction.

### SUMMARY OF THE INVENTION

The invention relates to a method for amplifying RNA in a sample, comprising: synthesizing single-stranded cDNA by incubating the sample RNA with reverse transcriptase and an oligonucleotide primer that primes synthesis in a direction toward 5′ end of the RNA; converting the single-stranded cDNA into double-stranded cDNA to form a transcription sample containing a cDNA template; eliminating single-stranded oligonucleotide from the transcription sample; and transcribing the cDNA template into RNA using an RNA polymerase. The RNA polymerase is preferably T7 RNA polymerase, T3 RNA polymerase, or Sp6 RNA polymerase, more preferably T7 RNA polymerase. The oligonucleotide primer is preferably T7dT₂₁ primer.

In one preferred embodiment, the eliminating comprises digesting the single-stranded oligonucleotide with at least one exonuclease, such as exonuclease I, RecJ_{f}, exonuclease T, or exonucease VII, preferably exonuclease I, exonuclease VII, or a combination thereof, more preferably an aqueous solution of exonuclease I and exonuclease VII. The method may further comprise heat-killing the exonuclease after the digesting.

In another preferred embodiment, the eliminating comprises hybridizing the single-stranded oligonucleotide with a complementary oligonucleotide.

In preferred embodiments, the RNA in the sample is a plurality of different RNA sequences in a tissue sample. Alternatively, the RNA in the sample is a single RNA sequence. In other preferred embodiments, the sample contains total RNA or mRNA from mammalian cells. Also, the RNA in the sample may be mRNA derived from a eukaryotic population of cells. In especially preferred embodiments, the sample is obtained by laser-capture microdissection (LCM).

In preferred embodiments, the method further comprises subjecting the transcribed RNA to a second round of amplification, preferably after purifying the transcribed RNA.

In some preferred embodiments, the method also comprises labeling the transcribed RNA with a label, e.g., a fluorescent, radioactive, enzymatic, hapten, biotin, digoxigenin, or aminoallyl label. Alternatively, the method further comprises synthesizing labeled cDNA from the transcribed RNA.

### BRIEF DESCRIPTION OF TEE DRAWINGS

FIG. 1 shows an agarose gel image of T7 RNA polymerase transcription reactions in the absence of a functional template, and in the presence of: lanes 1-3, T7dT₂₁ oligonucleotide; lanes 4-6, oligo-dT₁₂₋₁₈; lanes 7-9, hairpin T7 oligonucleotide; lanes 10-12, T7dT₂₁ and 1 kb DNA ladder; lanes 13-15, 1 kb ladder; lanes 16-18, aRNA from negative control reaction after two rounds of T7 RNA amplification. The ladder on each side of the gel is a 1 kb double-stranded DNA ladder with lengths ranging from 500 bp to 12,200 bp.

FIG. 2 shows the results of agarose gel electrophoresis of RNA products from T7 RNA transcription reactions in the absence of a functional double-stranded template, and in the presence of: lanes 1-3, scrambled oligo; lanes 4-6, dA₂₀ oligo; lanes 7-9, T7dT₂₁ oligo; lanes 10-12 T7dT₂₁ plus dA₂₀.

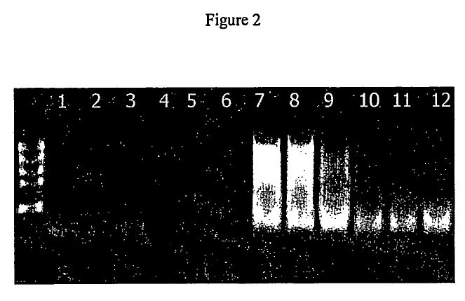

FIG. 3 shows a gel image of RNA products from two rounds of T7 RNA amplification. Lanes: 1-2, negative controls without exonuclease treatment; 3-4, 2 ng total RNA, no exonuclease treatment; 5-6, negative controls, exonuclease I treated; 7-8, 2 ng total RNA, exonuclease I treated; 9-10, negative controls, exonuclease VII treated; 11-12, 2 ng total RNA, exonuclease VII treated; 13-14, negative controls, exonuclease I and VII treated; 15-16, 2 ng total RNA, exonuclease I and VII treated.

FIG. 4 shows a gel image of RNA products. Lanes: 1-2, negative controls without exonuclease treatment; 3-4, 2 ng total RNA, no exonuclease treatment; 5-6, negative controls, treated with 20 U exonuclease I and 10 U exonuclease VII; 7-8, 2 ng total RNA, treated with 20 U exonuclease I and 10 U exonuclease VII; 9-10, negative controls, treated with 10 U exonuclease I and 5 U exonuclease VII; 11-12, 2 ng total RNA, treated with 10 U exonuclease I and 5 U exonuclease VII; 13-14, negative controls, treated with 2 U exonuclease I and 1 U exonuclease VII; 15-16, 2 ng total RNA treated with 2 U exonuclease I and 1 U exonuclease VII.

### DETAILED DESCRIPTION OF THE INVENTION AND PREFERRED EMBODIMENTS

The present invention provides methods for improving the preparation of templates for RNA polymerase transcription using enzymes such as, but not limited to, T7 RNA polymerase, T3 RNA polymerase and Sp6 RNA polymerase. Solutions containing transcription templates, which are usually double-stranded DNA, will frequently be contaminated by single-stranded oligonucleotides. This may be a result of carry-over of the cDNA synthesis primer if RNA was used as the starting template.

Oligonucleotide primers for use in the methods of the present invention can be of any suitable size. The oligonucleotide primers can be DNA, chimeric mixtures or derivatives or modified versions thereof, so long as they are still capable of priming the desired reaction. The oligonucleotide primer can be modified at the base moiety, sugar moiety, or phosphate backbone, and may include other appending groups or labels, so long as it is still capable of priming the desired amplification reaction. The oligonucleotide primers may be derived by cleavage of a larger nucleic acid fragment using non-specific nucleic acid cleaving chemicals or enzymes or site-specific restriction endonucleases, or by synthesis by standard methods known in the art, e.g. by use of an automated DNA synthesizer (such as are commercially available from Biosearch and Applied Biosystems) and standard phosphoramidite chemistry.

The presence of single-stranded oligonucleotide, in particular if the oligonucleotide contains a stretch of thymidine bases, will result in non-template derived production of RNA by RNA polymerases such as T7 RNA polymerase and T3 RNA polymerase.

In the present invention, single-stranded oligonucleotide is removed from the sample prior to transcription by either enzymatic digestion of single-stranded DNA by an exonuclease or by hybridization with a complementary oligonucleotide.

An “exonuclease” is an enzyme capable of digesting DNA or RNA from a free end, either 3′ or 5′. In the present invention, a preferred exonuclease is a single-strand specific exonuclease that is capable of digesting DNA. Examples of suitable exonucleases include, but not limited to, exonuclease I, RecJ_{f}, exonuclease T, exonuclease VII. These enzymes are commercially available from vendors such as New England Biolabs (Beverly, Mass.) and USB (Cleveland, Ohio). These enzymes may be used alone or in combination. A preferred combination is exonuclease I and exonuclease VII.

Transcription templates for RNA polymerase transcription may be generated from RNA by a suitable technique known in the art. The RNA used as the starting material may be complex, for example representing all the RNAs expressed in a tissue sample, or simple, such as RNA with one single sequence. By way of example but not limitation: cDNA is generated from RNA using a cDNA synthesis oligonucleotide containing a stretch of deoxythymidine bases at the 3′-end to prime reverse transcription and an RNA polymerase promoter site in the 5′-end, a reverse transcriptase and incubation in conditions conducive to reverse transcription. The single-stranded cDNA is then converted into double-stranded cDNA using a DNA polymerase, primed either by residual RNA oligomers from the RNA-cDNA hybrid, or by exogenous primers, such as random primers. The double-stranded cDNA template may be purified prior to transcription.

In the present invention one or more exonucleases are added to the double-stranded template prior to RNA polymerase transcription. An exonuclease, preferably exonuclease I or a combination of exonucleases, preferably exonuclease I or exonuclease VII, is added to the transcription template and incubated, preferably at a temperature of from 25 to 40° C. or more preferably at a temperature of about 37° C. for a period of from 1 to 60 minutes, preferably 2-35 minutes, or more preferably 4 to 20 minutes. The volume of the reaction is preferably 5-200 μl. The amount of exonuclease added is preferably, for exonuclease VII, 0.1 to 50 units, more preferably 0.2 to 20 units, or even more preferably 0.5 to 10 units per reaction and, for exonuclease I, preferably is from 0.1 to 200 units, more preferably 0.5 to 50 units, or even more preferably 1 to 20 units per reaction. After incubation, the exonucleases are inactivated by heat killing, e.g., at 55-100° C., preferably 65-90° C., or more preferably 70-80° C. for a sufficient time, e.g., 30 seconds to 20 minutes, preferably 1 to 10 minutes, or more preferably 2 to 5 minutes. Alternatively, exonucleases may be removed by purification of the double-stranded cDNA using by way of example but not limitation silica based DNA purification spin columns, such as PCRquick (Qiagen, Alameda, Calif.) or centrifugal filter membranes such as Microcon YM-100 (Millipore, Bedford, Mass.).

The transcription template may subsequently be transcribed using an appropriate RNA polymerase. A suitable enzyme known in the art is T7 RNA polymerase. A kit, such as the Ampliscribe kit, commercially available from Ambion (Austin, Tex.), can be used. An exemplary reaction contains ribonucleotides, some or all of which may be modified with a label (e.g., fluorescent, radioactive, enzymatic, biotin, or a hapten for antibody binding, or an aminoallyl to facilitate dying), a suitable buffer for RNA transcription, and an RNA polymerase such as T7 RNA polymerase. The reaction is incubated for a suitable period of time, usually 1 to 12 hours. The resulting RNA may be purified using one or more suitable purification methods and then used in numerous applications. By way of example, but not limitation, the amplified RNA may be used for hybridization to micro- and macro-arrays, library construction, library screening, RT-PCR, RNA interference experiments, as a probe in in situ hybridization experiments, hybridization to microsphere based arrays, such as the Luminex xMAP system (Luminex, Austin, Tex.) or BeadArray from Illumina (Illumina, San Diego, Calif.) and for expression of mRNAs in oocyte injections.

In another embodiment single-stranded oligonucleotides are removed prior to RNA polymerase transcription by adding a complementary oligonucleotide. The added oligonucleotide will hybridize to the first oligonucleotide, thereby rendering it double-stranded. The length of the second oligonucleotide is preferably similar to the length of the first oligonucleotide, but not necessarily identical. The second oligonucleotide may be DNA, RNA or a DNA/RNA chimera. It may contain a modified backbone such as phosphorothioate and locked DNA. It may contain modified bases, such as biotin, digoxigenin or dinitrophenyl. The concentration of the added oligonucleotide may be 1 to 10,000 times that of the first oligonucleotide, preferably 1 to 1,000 times, or more preferably 1 to 100 times that of the first oligonucleotide.

The following examples are provided to further illustrate the present invention and some of its embodiments and advantages.

### EXAMPLES

### Example 1

### T7 RNA Transcription Reaction without a Double-Stranded T7 RNA Polymerase Promoter-Containing Template

All transcription reactions were performed without T7 promoter-containing double-stranded DNA templates. Reactions contained 2 μl 10×T7 transcription buffer, 1.5 μl each of ATP, CTP, GTP and UTP, 2 μl of 0.1 M dithiothreitol, 2 μl of T7 RNA polymerase, all from the Ampliscribe T7 transcription kit (Ambion, Austin, Tex.) and 100 ng polyinosinic acid (Sigma) in a total volume of 25 μl. Reactions were incubated at 42° C. for 3 h. After incubation, 1 μl DNase I was added to each sample, which was then incubated at 37° C. for 15 min. 100 ng of polyinosinic acid was added to each reaction. The reactions were subsequently purified with an Rneasy kit (Qiagen, Alameda, Calif.). 6 μl ( 1/15^{th} of the volume) of the RNA products were run on a 1% agarose gel containing 1 M urea, stained with ethidium bromide and visualized under UV light.

In different reactions, in triplicates, the following additions to the T7 RNA transcription mix were tested: (1) 1 μg of T7dT₂₁ primer, i.e., 5′-TCTAGTACCTGCTTCACTGCATCTAATACGACTCACTATAGGGAGATTTTTTTT TTTTTTTTTTTTT-3′ (SEQ ID NO:1); (2) 1 μg of oligo-dT₁₂₋₁₈ (Amersham Biosciences), which is a mix of the oligos 5′-TTTTTTTTTTTT-3′ (SEQ ID NO:2), 5-TTTTTTTTTTTTT-3′ (SEQ ID NO:3), 5′-TTTTTTTTTTTTTT-3′ (SEQ ID NO:4), 5′-TTTTTTTTTTTTTTT-3′ (SEQ ID NO:5), 5′-TTTTTTTTTTTTTTTT-3′ (SEQ ID NO:6), 5′-TTTTTTTTTTTTTTTT-3′ (SEQ ID NO:7), and 5′-TTTTTTTTTTTTTTTTTT-3′ (SEQ ID NO:8); (3) 1 μg of T7 hairpin oligo, i.e., 5′-TTCCAGTGAGTCGTATCTAAAACTAATACGACTCACTATAGGGAGATTTTTTTT TTTTTTTTTTTTT-3′ (SEQ ID NO:9), which has an added sequence in the 5′-end to form a hairpin in which the T7 promoter site is double-stranded but mismatched; (4) 1 μg of T7dT₂₁ (SEQ ID NO:1) plus 20 ng of a 1 kb DNA ladder (Invitrogen); (5) 20 ng of a 1 kb DNA ladder; (6) 20 ng of aRNA produced in a negative control sample subjected to two rounds of T7 RNA amplification.

T7dT₂₁ was tested to see if the presence of free T7dT₂₁ oligonucleotide in the RNA transcription reaction mixture would result in the production of RNA in the absence of a template. Oligo-dT₁₂₋₁₈ was tested to see if the non-template derived production of RNA, if seen with T7dT₂₁, depended on the presence of a functional T7 RNA promoter site. The T7 hairpin oligonucleotide was tested to see if disruption of the T7 RNA polymerase promoter site would inhibit the non-template derived production of RNA. Double-stranded DNA was tested in the presence of T7dT₂₁ to see if double-stranded DNA could suppress the non-template derived production of RNA, if seen with T7dT₂₁. Double-stranded was tested on its own to check if it would promote non-template derived RNA production. Finally, aRNA produced in a negative control reaction after two cycles of T7 RNA amplification was tested to see if replicative forms of RNA are produced by T7 RNA polymerase (Biebricher and Luce, Embo J (1996) 15:3458).

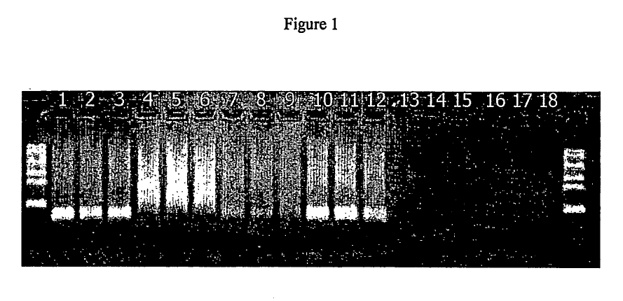

FIG. 1 shows an agarose gel image of the RNA products. The addition of T7dT₂₁ into the transcription reaction mix resulted in the production of RNA, evident as a smear ranging from high molecular weights to low molecular weights. There was also a distinct low molecular weight band at less than 500 bp. The production of RNA ranging in size from high to low molecular weights was not dependent on a functional T7 RNA promoter site, as the addition of oligo-dT₁₂₋₁₈ produced the same pattern. However, the low molecular weight band was absent when oligo-dT₁₂₋₁₈ was added to the RNA transcription reaction. The hairpin T7 oligonucleotide did not significantly reduce the production of RNA with a range from high to low molecular weight, but it did reduce the formation of the low molecular weight band. The addition of double-stranded DNA neither inhibited the production of template independent RNA promoted by T7dT₂₁, nor promoted RNA production on its own. Finally, RNA produced in a negative control sample from a previous two-round T7 RNA amplification reaction did not promote the production of RNA when spiked into the T7 RNA transcription mix, arguing against the formation of replicative RNA in the T7 RNA transcription reaction under these conditions.

This experiment showed that T7 RNA polymerase can produce RNA in the absence of a functional template, i.e., a template with a double-stranded T7 RNA polymerase promoter site. The reaction is promoted by the presence of single-stranded nucleic acid, in particular single-stranded oligonucleotides. We have shown that a stretch of single-stranded deoxythymidine bases, such as those present in oligo-dT₁₂₋₁₈ or T7dT₂₁ oligonucleotides, is sufficient to promote this reaction. The RNA produced has a range of molecular weights from very high, >24,000 nucleotides, to low, less than 1000 nucleotides. When the T7 RNA polymerase promoter site was present in the oligonucleotide, a distinct low molecular weight band was produced, which was reduced when the T7 promoter site was disrupted by a mismatch duplex.

### Example 2

### T7 RNA Transcription in the Presence of Single- and Double-Stranded Oligonucleotides

All transcription reactions were performed without double-stranded T7 promoter-containing DNA templates. Reactions contained 2 μl 10×T7 transcription buffer, 1.5 μl each of ATP, CTP, GTP and UTP, 2 μl of 0.1 M dithiothreitol, 2 μl of T7 RNA polymerase all from the Ampliscribe T7 transcription kit (Ambion, Austin, Tex.) in a total volume of 25 μl. Reactions were incubated at 42° C. for 3 hrs. After incubation, 0.5 μl DNase I was added to an aliquot (7 μl) of each sample, which was then incubated at 37° C. for 15 min. The entire aliquot was then run on a 1% agarose gel containing 1 M urea, stained with ethidium bromide and visualized under UV light.

In triplicate reactions, the following additions were done to the T7 RNA transcription mix: (1) 1 μg of a 28-mer oligonucleotide with a scrambled sequence: 5′-GCTGACTCGTACTCGAGTTAGTGGTAGT-3′ (SEQ ID NO:10); (2) 1 μg of a dA₂₀ oligonucleotide (20 deoxyadenine bases, SEQ ID NO:11); (3) 1 μg of T7dT₂₁ oligonucleotide (SEQ ID NO:1); (4) 1 μg of T7dT₂₁ oligonucleotide (SEQ ID NO:1) plus 1 μg of a dA₂₀ oligonucleotide, i.e., 5′-AAAAAAAAAAAAAAAAAAAA-3′ (SEQ ID NO:11).

We have shown that the ability of a single-stranded oligonucleotide to promote the production of RNA, in the absence of a functional template, in a T7 RNA transcription reaction is sequence dependent. The scrambled oligo resulted in a very low amount of RNA produced and the dA₂₀ oligo did not result in any detectable RNA production, whereas the T7dT₂₁ oligo caused a significant production of RNA, of a wide range of lengths. This reaction is template-independent in the sense that there is no classical template present, i.e., a polynucleotide with a double-stranded promoter for T7 RNA polymerase. However, this does not rule out that T7 RNA polymerase uses the oligonucleotides as a template for RNA polymerization. We have also shown that rendering the stretch of deoxythymidine bases at the 3′-end of the T7dT₂₁ oligonucleotide, double-stranded by hybridization to a 20-base deoxyadenine oligonucleotide, dramatically reduces the production of RNA caused by T7dT₂₁. Therefore, this is a template-independent, sequence-dependent and single-strand-dependent reaction. The addition of dA₂₀ provides a method for inhibiting this reaction.

### Example 3

### Using Exonucleases to Limit T7dT₂₁ Primer-Generated RNA Production

Two rounds of T7 RNA amplification, starting from 2 ng of total RNA and blank negative controls, were used to test the efficacy of exonuclease digestion of single-stranded oligonucleotides prior to T7 RNA transcription in the first round, for eliminating RNA production in the negative control reactions. RNA produced in the negative control reactions is referred to herein as “background RNA.” In the course of improving the T7 RNA amplification system we have learned that the production of background RNA does not appear to be dependent on exogenous contamination. Rather, it appears to be dependent on the amount of T7dT₂₁ primer present in the T7 RNA transcription mix prior to transcription.

Four conditions were tested: no exonucleases, exonuclease I, exonuclease VII, and finally a mixture of exonuclease I and VII. Each condition contained two positive samples, which were 2 ng of total rat brain RNA, and two negative samples that contained no RNA. All samples also contained 100 ng of polyinosinic acid (Sigma).

First Round:

First-strand cDNA synthesis: To each sample, 50 ng of T₇dT₂₁ primers (SEQ ID NO:1) were added. The mixture was heated at 70° C. for 10 minutes and then put on ice. The first-strand cDNA synthesis was performed with 100 units of Superscript II reverse transcriptase (Invitrogen) in a volume of 10 μl for 2 hours at 42° C. The reaction contained 50 mM Tris-HCl, 75 mM KCl, 3 mM MgCl₂, 10 mM dithiothreitol, 500 μM dNTPs and 20 units RNasin (Promega). The reaction was terminated by heating at 70° C. for 10 min.

Second-strand cDNA synthesis: Second strand cDNA was synthesized by adding 4 μl 10×Bst polymerase buffer (200 mM Tris-HCl pH 8.8, 100 mM KCl, 100 mM (NH₄)₂SO₄, 20 mM MgSO₄, 1% Triton X-100), 1.5 μl of 10 mM dNTPs, 12 U of Bst DNA polymerase large fragment (New England Biolabs, Beverly, Mass.), 2.5 U of thermostable RNase H (Epicentre, Madison, Wis.) in a total volume of 40 μl. The mixtures were incubated at 65° C. for 10 min, followed by heating at 80° C. for 10 min to terminate DNA synthesis. The samples were then subjected to four sets of treatment: (1) no addition of exonucleases; (2) 20 units of exonuclease I (New England Biolabs) was added, and the mix was incubated 10 min at 37° C., followed by heating at 80° C. for 10 min; (3) 10 units of exonuclease VII (USB, Cleveland, Ohio) was added to the reaction, incubated 10 min at 37° C. and followed by heating at 80° C. for 10 min; (4) 20 units of exonuclease I and 10 units of exonuclease VII were added to the reaction, incubated 10 min at 37° C. and followed by heating at 80° C. for 10 min.

To every reaction 100 ng of polyinosinic acid and 200 μl of PB buffer (Qiagen) were added and the mix was purified on a PCRquick purification column (Qiagen) according to the manufacturer's directions. The DNA was eluted in 30 μl 1 mM Tris-HCl pH 8.0.

The purified double-stranded cDNA was dried down to 16 μl in a SpeedVac, and then transcribed with T7 RNA polymerase. In a total volume of 40 μl, 4 μl 10×T7 transcription buffer, 3 μl each of ATP, CTP, GTP and UTP, 4 μl 0.1M dithiothreitol and 4 μl of T7 RNA polymerase were used. All reagents in the transcription reaction were from Epicentre's Ampliscribe T7 Transcription kit. The transcription reaction was carried out for 3 hours at 42° C., and followed by Dnase I (2 μl) treatment for 15 min at 37° C. Polyinosinic acid (100 ng) was added to the samples prior to purification with Qiagen's Rneasy kit. The eluted RNA was dried down to 8 μl in a SpeedVac.

Second Round:

To each sample, 0.5 μg of random hexamers (Amersham Biosciences) was added. The mix was denatured at 70° C. for 10 min, and cooled on ice. cDNA synthesis was performed as above, except incubation was done at 37° C. for 1 h. 0.5 μl of Rnase H (Epicentre) was added to the first-strand reaction, and incubated at 37° C. for 20 min. The reaction was terminated by heating at 95° C. for 2 min and put on ice. Subsequently, 250 ng of T7dT₂₁ primer was added to the reaction, heated at 70° C. for 5 min then 42° C. for 10 min. Second-strand cDNA was synthesized using E. Coli DNA polymerase I by adding 15 μl of 5× second-strand cDNA synthesis buffer (100 mM Tris-HCl pH 6.9, 23 mM MgCl₂, 450 mM KCl, 0.75 mM β-NAD+, 50 mM (NH₄)₂SO₄), 1.5 μl of 10 mM dNTPs, 20 U E. Coli DNA polymerase I (Invitrogen), and 1.1 U Rnase H (Invitrogen) in a final volume of 75 μl. The mixture was incubated at 37° C. for 10 min. 5 U of T4 DNA polymerase was then added to the mixture and incubated at 16° C. for 15 min. 100 ng of polyinosinic acid and 375 μl of PB buffer (Qiagen) were added to the reaction. The samples were purified on a PCR purification column (Qiagen) according to the manufacturer's directions. The DNA was eluted in 30 μl of 1 mM Tris-HCl pH 8.0. The double-stranded cDNA was dried down to 8 μl in a SpeedVac and transcribed with T7 RNA polymerase. In a volume of 25 μl reaction, 2 μl 10×T7 transcription buffer, 1.5 μl each of ATP, CTP, GTP and UTP, 2 μl 0.1 M dithiothreitol and 2 μl of T7 RNA polymerase were used. The transcription reaction was carried out for 3 hours at 42° C. and following by DNase I treatment (1 μl) for 15 min at 37° C. Reaction was purified using Qiagen's Rneasy kit. Aliquots (2 μl out of 48 μl) of the purified RNA products were analyzed on a 1% agarose gel containing 1 M urea.

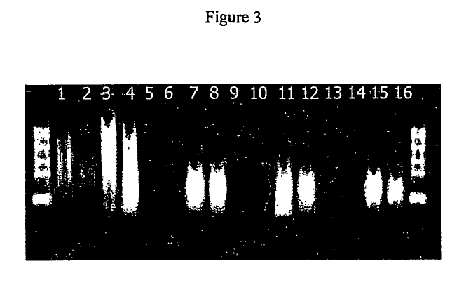

The gel image provided in FIG. 3 shows that two rounds of T7 RNA amplification resulted in the production of RNA in negative control reactions. This background RNA had a range of molecular weights from very high to a few hundred bases. Both exonuclease I and exonuclease VII were efficient in reducing the amount of background RNA. The combination of the exonucleases appeared to be the most efficient solution.

The data show that digestion of single-stranded oligonucleotides, in this case likely the T7dT₂₁ oligonucleotide remaining in the sample from the initial first-strand cDNA synthesis step, dramatically reduces the production of background RNA in two rounds of T7 RNA amplification. Elimination of background RNA in this procedure improves the purity of the amplified RNA by eliminating artifactual RNA from the amplified sample.

### Example 4

### Titration of Exonuclease I and VII for Limiting T7dT₂₁ Primer-Generated Background RNA

Two rounds of T7 RNA amplification, starting from 2 ng of total RNA and blank negative controls, were used to titrate the amount of exonucleases required to digest single-stranded oligonucleotides prior to T7 RNA transcription in the first round in order to eliminate background RNA production in the negative control reactions.

Three concentrations of a mixture of exonucleases were tested: (1) 20 and 10 U of exonuclease I and VII, respectively, per reaction; (2) 10 and 5 U of exonuclease I and VII respectively per reaction; (3) 2 and 1 U of exonuclease I and VII respectively per reaction. Each condition contained two positive samples, which were 2 ng of total rat brain RNA, and two negative samples that contained water. All samples also contained 100 ng of polyinosinic acid (Sigma).

First Round:

First-strand cDNA synthesis: To each sample, 50 ng of T₇dT₂₁ primers (5′-TCTAGTACCTGCTTCACTGCATCTAATACGACTCACTATAGGGAGATTTTTTTT TTTTTTTTTTTTT-3′, SEQ ID NO:1) was added. The mixture was heated at 70° C. for 10 min and then put on ice. The first-strand cDNA synthesis was performed with 100 units of Superscript II reverse transcriptase (Invitrogen) in a volume of 10 μl for 2 hours at 42° C. The reaction contained 50 mM Tris-HCl, 75 mM KCl, 3 mM MgCl₂, 10 mM dithiothreitol, 500 μM dNTPs and 20 units RNasin (Promega). The reaction was terminated by heating at 70° C. for 10 min.

Second-strand cDNA synthesis: Second-strand cDNA was synthesized by adding 4 μl 10×Bst polymerase buffer (200 mM Tris-HCl pH 8.8, 100 mM KCl, 100 mM (NH₄)₂SO₄, 20 mM MgSO₄, 1% Triton X-100), 1.5 μl of 10 mM dNTPs, 12 U of Bst DNA polymerase large fragment (New England Biolabs, Beverly, Mass.), and 2.5 U of thermostable RNase H (Epicentre, Madison, Wis.) in a total volume of 40 μl. The mixtures were incubated at 65° C. for 10 min, followed by heating at 80° C. for 10 min to terminate DNA synthesis. The samples were then subjected to four sets of treatment: (1) no addition of exonucleases; (2) 20 and 10 U of exonuclease I and VII, respectively, were added per reaction, and the mix was incubated 10 min at 37° C., followed by heating at 80° C. for 10 min; (3) 10 and 5 U of exonuclease I and VII, respectively, were added to the reaction, incubated 10 min at 37° C. and followed by heating at 80° C. for 10 min; (4) 2 and 1 U of exonuclease I and VII, respectively, were added to the reaction, incubated 10 min at 37° C. and followed by heating at 80° C. for 10 min.

To every reaction 100 ng of polyinosinic acid and 200 μl of PB buffer (Qiagen) were added and the mix was purified on a PCRquick purification column (Qiagen) according to the manufacturer's directions. The DNA was eluted in 30 μl 1 mM Tris-HCl pH 8.0.

The samples were transcribed in a total volume of 100 pt, containing 10 μl 10×T7 transcription buffer, 7.5 μl each of ATP, CTP, GTP and UTP, 10 μl 0.1M dithiothreitol and 6 μl of T7 RNA polymerase. All reagents in the transcription reaction were from Epicentre's Ampliscribe T7 Transcription kit. The transcription reaction was carried out for 3 h at 42° C., and followed by Dnase I (2 μl) treatment for 15 min at 37° C. Polyinosinic acid (100 ng) was added to the samples prior to purification with Qiagen's Rneasy kit. The eluted RNA was dried down to 4.5 μl in a SpeedVac.

Second Round:

To each sample, 0.5 μg of random hexamers (Amersham Biosciences) was added. The mix was denatured at 70° C. for 10 min, and cooled on ice. cDNA synthesis was performed as above, except incubation was done at 37° C. for 1 h. 0.5 μl of Rnase H (Epicentre) was added to the first-strand reaction, and incubated at 37° C. for 20 min. The reaction was terminated by heating at 95° C. for 2 min and put on ice. Subsequently, 250 ng of T7dT₂₁ primer was added to the reaction, heated at 70° C. for 5 min then 42° C. for 10 min. Second-strand cDNA was synthesized using E. Coli DNA polymerase I by adding 15 ill of 5× second-strand cDNA synthesis buffer (100 mM Tris-HCl pH 6.9, 23 mM MgCl₂, 450 mM KCl, 0.75 mM β-NAD+, 50 mM (NH₄)₂SO₄), 1.5 μl of 10 mM dNTPs, 20 U E. Coli DNA polymerase I (Invitrogen), and 1.1 U Rnase H (Invitrogen) in a final volume of 75 nil. The mixture was incubated at 37° C. for 10 min. 5 U of T4 DNA polymerase was then added to the mixture and incubated at 16° C. for 15 min. 100 ng of polyinosinic acid and 375 μl of PB buffer (Qiagen) were added to the reaction. The samples were purified on a PCR purification column (Qiagen) according to the manufacturer's directions. The DNA was eluted in 30 μl of 1 mM Tris-HCl pH 8.0. The DNA was concentrated to 16 μl and transcribed in a total volume of 40 μl, containing 4 μl 10×T7 transcription buffer, 3 μl each of ATP, CTP, GTP and UTP, 4 μl 0.1 M dithiothreitol and 4 μl of T7 RNA polymerase. After transcription, the samples were incubated with 1 μl DNase I for 15 min at 37° C. The resulting RNA was purified using an Rneasy kit. Aliquots of the purified RNA products, 2 μl out of 48 μl, were analyzed on a 1% agarose gel containing 1 M urea.

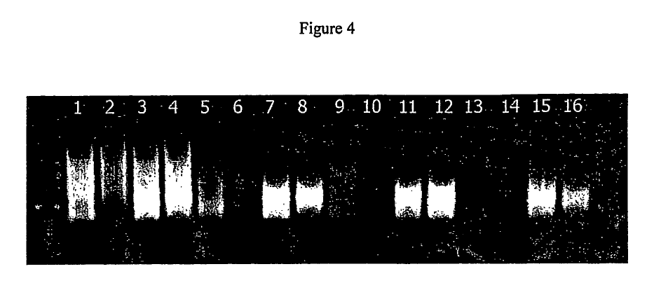

The gel image in FIG. 4 shows that 2 and 1 U of exonuclease I and VII, respectively, effectively reduced the production of background RNA in negative controls.

### Example 5

### Two-Round aRNA Amplification of LCM Sample

RNA Extraction from LCM Samples:

A sample obtained by laser-capture microdissection (LCM) sample is put into 10 μl of RLT/β-ME solution containing 200 ng polyinosinic acid (Sigma, Saint Louis, Mo.). The RLT/β-ME solution is prepared by adding 10 μl of β-ME to each ml of RLT (Qiagen, Valencia, Calif.). The sample is incubated at 42° C. for 20 min and chilled on ice. Ethanol (100%, 10 μl) is added to the sample and mixed briefly. The sample is left on ice for 10 min. A Zymo-Spin Column (Zymo research, Orange, Calif.) is placed into a 2-ml collection tube and the sample mixture is transferred to the column. The column with the tube is spun at full speed in a microcentrifuge for 15 sec. The column is washed twice by adding RPE (200 μl) to the column followed by centrifugation at full speed for 1 min. The column is placed into a new 1.5 ml tube, 10 μl of water is then directly to the membrane of Zymo-Spin Column. After 5 min, RNA is eluted by spinning the column at full speed for 1 min. The RNA eluate is adjusted to 4 μl by speed vacuum.

First Round of aRNA Amplification:

First-strand cDNA synthesis: Fifty ng of T7dT₂₁ primer (5′-TCTAGTACCTGCTTCACTGCATCTAATACGACTCACTATAGGGAGATTTTTTT TTTTTTTTTTTTTT-3′ (SEQ ID NO:1), PAGE purified, Qiagen) is added to each sample. The sample mixture is heated at 65° C. for 5 min and then chilled on ice. The first-strand cDNA synthesis is performed with 100 units of Superscript II reverse transcriptase (Invitrogen, Carlsbad, Calif.) in a volume of 10 μl for 2 hours at 42° C. The reaction contains 50 mM Tris-HCl, 75 mM KCl, 3 mM MgCl₂, 10 mM dithiothreitol, 500 μM dNTPs (MBI Fermentas, Hanover, Md.) and 20 units RNasin (Promega, Madison, Wis.).

Second-strand cDNA synthesis: Second-strand cDNA is synthesized in a total volume of 20 μl for 10 min at 65° C. To the first-strand cDNA (10 μl), 1 μl 10× thermopol buffer (200 mM Tris-HCl pH 8.8, 100 mM KCl, 100 mM (NH₄)₂SO₄, 20 mM MgSO₄, 1% Triton X-100), 1 μl of 10 mM dNTPs, 8 U of Bst DNA polymerase large fragment (New England Biolabs, Beverly, Mass.), and 2.5 U of thermostable RNase H (Epicentre, Madison, Wis.) are added. The mixture is heated to 80° C. for 3 min, and 4 units of exonuclease I (New England Biolabs) and 2 units of exonuclease VII (USB, Cleveland, Ohio) are added to the reaction and incubated for 10 min at 37° C. The reaction is heated to 80° C. for 3 min and chilled on ice. This product is then added, unpurified, into the subsequent transcription reaction.

Transcription with T7 RNA polymerase: In a total volume of 100 μl, 8 μl 10×T7 transcription buffer, 6 μl each of ATP, CTP, GTP and UTP, 8 μl 0.1M dithiothreitol and 8 μl of T7 RNA polymerase are used. All reagents in the transcription reaction are obtained from Epicentre's Ampliscribe T7 Transcription kit (Epicentre). The transcription reaction is carried out for 3 h at 42° C. followed by Dnase I (4 μl) treatment for 15 min at 37° C.

aRNA Purification:

Polyinosinic acid (100 ng/μl, 1 μl), RLT/β-ME (350 μl), and 100% EtOH (250 μl) are added to the sample. A Zymo-spin column is placed in a collection tube and the sample mixture is transferred to the column. The column is centrifuged at full speed (≧10,000 g) for 10-15 seconds, and the flow-through is discarded. The column is washed twice by adding 700 μl of RPE to the column followed by spinning at full speed for 15-60 seconds. aRNA is eluted by directly adding 10 μl of Rnase-free water to the column matrix and spinning at full speed for 1 min. The eluate is dried down to 4 μl for second-round amplification.

Second-Round of aRNA Amplification:

To each sample, 0.5 μg of random hexamers (Amersham Biosciences, Piscataway, N.J.) is added. The mixture is denatured at 65° C. for 5 min and chilled on ice. cDNA synthesis is carried out as described above, except incubation is performed at 37° C. for 1 h. Rnase H (0.5 μl, Epicentre) is added to the first-strand reaction for 20 min at 37° C. The reaction is terminated by heating at 95° C. for 2 min and then chilled on ice. Subsequently, 250 ng of T7dT₂₁ primer is added to the reaction, which is first heated at 70° C. for 5 min and then incubated at 42° C. for 10 min. Second-strand cDNA is synthesized using E. Coli DNA polymerase I by adding 3 μl of 10× reaction buffer (500 mM Tris-HCl pH 7.5, 100 mM MgCl₂, 10 mM DTT), 1.5 μl of 10 mM dNTPs, 20 U E. Coli DNA polymerase I (Fermentas), and 5 U Rnase H (Epicentre) in a final volume of 40 μl. The mixture is incubated at 37° C. for 10 min followed by heating to 80° C. for 3 min. The double-stranded cDNA template is transcribed by adding 8 μl 10×T7 transcription buffer, 6 μl each of ATP, CTP, GTP and UTP, 8 μl 0.1 M dithiothreitol, and 8 μl of T7 RNA polymerase (Epicentre) in a total volume of 100 μl. The transcription reaction is carried out for 3 hr at 42° C. followed by DNase I treatment (4 μl) for 15 min at 37° C. Reaction is purified by using Qiagen's Rneasy kit, and an aliquot (2 μl out of 48 μl) of the purified RNA products is analyzed on a 1% agarose gel containing 1 M urea.

While the above detailed description and preferred embodiments and examples have been provided to illustrate the invention and its various features and advantages, it will be understood that invention is defined not by the foregoing, but by the following claims as properly construed under principles of patent law.

What is claimed is:

1. A method for amplifying RNA in a sample, comprising:
synthesizing single-stranded cDNA by incubating the sample RNA with reverse transcriptase and an oligonucleotide primer that primes synthesis in a direction toward 5′ end of the RNA;
converting the single-stranded cDNA into double-stranded cDNA to form a transcription sample containing a cDNA template;
eliminating single-stranded oligonucleotide from the transcription sample; and
transcribing the cDNA template into RNA using an RNA polymerase.

2. A method as defined in claim 1, wherein said eliminating comprises digesting the single-stranded oligonucleotide with at least one exonuclease.

3. A method as defined in claim 2, wherein the exonuclease is exonuclease I, RecJ_{f}, exonuclease T, or exonuclease VII.

4. A method as defined in claim 2, wherein the exonuclease is exonuclease I, exonuclease VII, or a combination thereof.

5. A method as defined in claim 2, further comprising heat-killing the exonuclease after the digesting.

6. A method as defined in claim 1, wherein said eliminating comprises hybridizing the single-stranded oligonucleotide with a complementary oligonucleotide.

7. A method as defined in claim 1, wherein the RNA polymerase is T7 RNA polymerase, T3 RNA polymerase, or Sp6 RNA polymerase.

8. A method as defined in claim 1, wherein the RNA in the sample is a plurality of different RNA sequences in a tissue sample.

9. A method as defined in claim 1, wherein the RNA in the sample is a single RNA sequence.

10. A method as defined in claim 1, wherein the oligonucleotide primer is T7dT₂₁ primer (SEQ ID NO:1).

11. A method as defined in claim 1, further comprising:
subjecting the transcribed RNA to a second round of amplification.

12. A method as defined in claim 11, further comprising:
purifying the transcribed RNA before the second round of amplification.

13. A method as defined in claim 11, wherein said eliminating comprises digesting the single-stranded oligonucleotide with at least one exonuclease selected from the group consisting of exonuclease I, RecJ_{f}, exonuclease T, exonuclease VII, and combinations thereof.

14. A method as defined in claim 13, wherein the RNA polymerase is T7 RNA polymerase.

15. A method as defined in claim 11, wherein said eliminating comprises digesting the single-stranded oligonucleotide with an aqueous solution of exonuclease I and exonuclease VII.

16. A method as defined in claim 1, wherein the sample contains total RNA or mRNA from mammalian cells.

17. A method as defined in claim 1, wherein the sample is obtained by laser-capture microdissection.

18. A method as defined in claim 1, further comprising labeling the transcribed RNA with a label or synthesizing labelled cDNA from the transcribed RNA.

19. A method as defined in claim 1, further comprising labeling the transcribed RNA with a fluorescent, radioactive, enzymatic, hapten, biotin, digoxigenin, or aminoallyl label.

20. A method as defined in claim 1, wherein the RNA in the sample is mRNA derived from a eukaryotic population of cells.
